SEQUENCE LISTING (25 sequences)

SEQ ID NO: 1 - DNA, 25 bp, Artificial Sequence
  Oligonucleotide primer zc45814
gttgccatgtagaccccttgaagag

SEQ ID NO: 2 - DNA, 25 bp, Artificial Sequence
  Oligonucleotide primer zc45815
accagccccagcaagagcacaagag

SEQ ID NO: 3 - DNA, 25 bp, Artificial Sequence
  Oligonucleotide primer  zc45816
tgacttcaacagcgacacccactcc

SEQ ID NO: 4 - DNA, 25 bp, Artificial Sequence
  Oligonucleotide primer  zc45811
acagaattattccagggtttatgtg

SEQ ID NO: 5 - DNA, 25 bp, Artificial Sequence
  Oligonucleotide primer  zc45812
gcagtatcctagaatctttgtgctc

SEQ ID NO: 6 - DNA, 25 bp, Artificial Sequence
  Oligonucleotide primer zc45813
tgagaacttcatcctaaagcatacg

SEQ ID NO: 7 - DNA, 25 bp, Artificial Sequence
  Oligonucleotide primer  zc45628
gcccatgctcctcacctgtattttg

SEQ ID NO: 8 - DNA, 25 bp, Artificial Sequence
  Oligonucleotide primer zc45629
actcggagaattgtgcaggtgtcat

SEQ ID NO: 9 - DNA, 25 bp, Artificial Sequence
  Oligonucleotide primer zc45630
ccagttactatgagtgaaagggagc

SEQ ID NO: 10 - DNA, 25 bp, Artificial Sequence
  Oligonucleotide primer zc45631
gcctgggttttgtggtcatctattc

SEQ ID NO: 11 - DNA, 25 bp, Artificial Sequence
  Oligonucleotide primer zc45632
gacgagagtgctggggaataaaaag

SEQ ID NO: 12 - DNA, 25 bp, Artificial Sequence
  Oligonucleotide primer  zc45633
tatcagaagcccattattcagagcg

SEQ ID NO: 13 - DNA, 25 bp, Artificial Sequence
  Oligonucleotide primer  zc47406
agcaaaatacaaagcctaagatgag

SEQ ID NO: 14 - DNA, 25 bp, Artificial Sequence
  Oligonucleotide primer  zc47407
tgggcggattgttgtttaacttgta

SEQ ID NO: 15 - DNA, 25 bp, Artificial Sequence
  Oligonucleotide primer  zc47408
gttagaaaagtaagaagcagtcaat

SEQ ID NO: 16 - DNA, 35 bp, Artificial Sequence
  Oligonucleotide primer  zc52566
cagccgaagtggaggtgatagcattgctttcgtgt

SEQ ID NO: 17 - DNA, 35 bp, Artificial Sequence
  Oligonucleotide primer  zc52567
cagccgaagtcccttatgttgtgctgtatcctgtg

SEQ ID NO: 18 - DNA, 35 bp, Artificial Sequence
  Oligonucleotide primer  zc52568
cagccgaagtgttgccatgtagaccccttgaagag

SEQ ID NO: 19 - DNA, 35 bp, Artificial Sequence
  Oligonucleotide primer  zc52569
cagccgaagtgcctgggttttgtggtcatctattc

SEQ ID NO: 20 - DNA, 35 bp, Artificial Sequence
  Oligonucleotide primer  zc52570
cagccgaagttgggcggattgttgtttaacttgta

SEQ ID NO: 21 - DNA, 35 bp, Artificial Sequence
  Oligonucleotide primer  zc52571
cagccgaagtagcctccgcctcctgggttcaagtg

SEQ ID NO: 22 - DNA, 35 bp, Artificial Sequence
  Oligonucleotide primer  zc52572
cagccgaagtgcccatgctcctcacctgtattttg

SEQ ID NO: 23 - DNA, 35 bp, Artificial Sequence
  Oligonucleotide primer  zc52573
cagccgaagtagccaaggaagagtcggaggagtcg

SEQ ID NO: 24 - DNA, 35 bp, Artificial Sequence
  Oligonucleotide primer  zc52574
cagccgaagttgagattcctggttcgggtgttacg

SEQ ID NO: 25 - DNA, 35 bp, Artificial Sequence
  Oligonucleotide primer   zc52575
cagccgaagtcgagaatgtcaaggcaaagatccaa

(19) United States
(12) Patent Application Publication
(10) Pub. No.: US 2007/0298427 A1
(43) Pub. Date: Dec. 27, 2007

(54) METHODS OF GENE EXPRESSION ANALYSIS

(76) Inventor: Theodore E. Whitmore, Redmond, WA (US)

Correspondence Address: ZYMOGENETICS, INC.;INTELLECTUAL PROPERTY DEPARTMENT, 1201 EASTLAKE AVENUE EAST, SEATTLE, WA, 98102-3702, US

(21) Appl. No.: 11/766,652

(22) Filed: Jun. 21, 2007

Related U.S. Application Data
(60) Provisional application No. 60/805,533, filed on Jun. 22, 2006.

(51) Int. Cl. C12Q 1/68

(52) U.S. Cl. 435/6

(57) ABSTRACT

The present invention relates to methods for cost effective, high throughput, multiplexed nucleic acid (gene) expression analysis using photobiotin-labeled anti-sense RNA (aRNA) as hybridization probes in conjunction with a multiplex capable system. The use of the relatively inexpensive photobiotin provides a cost effective alternative to the use of currently available, expensive biotin analogs or other nonradioisotopic labeling methods.

### CROSS-REFERENCE TO RELATED APPLICATIONS

This application claims the benefit of U.S. Provisional Application Ser. No. 60/805,533, filed Jun. 22, 2006, which is herein incorporated by reference in its entirety.

### FIELD OF THE INVENTION

The present invention relates generally to methods of gene expression analysis using photobiotin labeled anti-sense RNA (aRNA).

### BACKGROUND OF THE INVENTION

In the biosciences, the introduction of nonradioistopic labeled nucleic acid probes has been essential for the development of large-scale multiplexed gene expression array technologies where hundreds or thousands of genes can be assayed in a single reaction.

At present, these can be grouped into two basic categories: Chip based arrays such as the Affymetrix GeneChip type microarrays (Fondor et al., “Multiplexed Biochemical Assays with Biological Chips,” Nature, 364:555-556 (1993) and U.S. Pat. No. 6,344,316 issued to Lockhart et al.) and microsphere (bead) based systems such as Luminex Corporation's xMAP® technology and Quantum Dot Corporation's mosaic gene expression assay system (Yang et al., “BADGE, BeadsArray for the Detection of Gene Expression, a High-Throughput Diagnostic Bioassay,” Genome Res., 11:1888-1898 (2001), Naciff et al., “Design of a Microsphere-Based High-Throughput Gene Expression Assay to Determine Estrogenic Potential,” Environmental Health Perspectives, 113:1164-1171 (2005), and U.S. Pat. No. 6,599,331 and U.S. Pat. No. 6,632,526).

Unfortunately, these gene expression array technologies can be very costly and time consuming. Accordingly, there remains a need in the art for cost effective, high-throughput methods for the analysis of gene expression.

### DETAILED DESCRIPTION OF THE INVENTION

The present invention solves this need by providing a cost effective method for gene expression analysis using photobiotin labeling of aRNA. Specifically, the present invention relates to the nonradioistopic labeling of anti-sense RNA (aRNA) using photobiotin and its use for cost effective high throughput multiplexed nucleic acid (gene) expression analysis. This entails using biotin-labeled aRNA as a hybridization probe in conjunction with a multiplex capable system such as the Luminex® 100 fluidic type instrument and xMAP® color-coded microspheres (Luminex Corp., Austin, Tex.). The use of the relatively inexpensive photobiotin provides a cost effective alternative to expensive biotin analogs or other nonradioisotopic labeling methods.

A) Definitions

In the description that follows, a number of terms are used extensively. The following definitions are provided to facilitate understanding of the invention.

An array of oligonucleotides as used herein refers to a multiplicity of different (sequence) oligonucleotides attached (preferably through a single terminal covalent bond) to one or more solid supports where, when there is a multiplicity of supports, each support bears a multiplicity of oligonucleotides. The term “array” can refer to the entire collection of oligonucleotides on the support(s) or to a subset thereof. The term “same array” when used to refer to two or more arrays is used to mean arrays that have substantially the same oligonucleotide species thereon in substantially the same abundances. The spatial distribution of the oligonucleotide species may differ between the two arrays, but, in a preferred embodiment, it is substantially the same. It is recognized that even where two arrays are designed and synthesized to be identical there are variations in the abundance, composition, and distribution of oligonucleotide probes. These variations are preferably insubstantial and/or compensated for by the use of controls as described herein.

The phrase “massively parallel screening” refers to the simultaneous screening of at least about 100, preferably about 1000, more preferably about 10,000 and most preferably about 1,000,000 different nucleic acid hybridizations.

The terms “nucleic acid” or “nucleic acid molecule” refer to a deoxyribonucleotide or ribonucleotide polymer in either single- or double-stranded form, and unless otherwise limited, would encompass known analogs of natural nucleotides that can function in a similar manner as naturally occurring nucleotides.

An oligonucleotide is a single-stranded nucleic acid ranging in length from 2 to about 1000 nucleotides, more typically from 2 to about 500 nucleotides in length.

As used herein a “probe” is defined as an oligonucleotide capable of binding to a target nucleic acid of complementary sequence through one or more types of chemical bonds, usually through complementary base pairing, usually through hydrogen bond formation. As used herein, an oligonucleotide probe may include natural (i.e. A, G, C, or T) or modified bases (7-deazaguanosine, inosine, etc.). In addition, the bases in oligonucleotide probe may be joined by a linkage other than a phosphodiester bond, so long as it does not interfere with hybridization. Thus, oligonucleotide probes may be peptide nucleic acids in which the constituent bases are joined by peptide bonds rather than phosphodiester linkages.

The term “target nucleic acid” refers to a nucleic acid (often derived from a biological sample and hence referred to also as a sample nucleic acid), to which the oligonucleotide probe specifically hybridizes. It is recognized that the target nucleic acids can be derived from essentially any source of nucleic acids (e.g., including, but not limited to chemical syntheses, amplification reactions, forensic samples, etc.). It is either the presence or absence of one or more target nucleic acids that is to be detected, or the amount of one or more target nucleic acids that is to be quantified. The target nucleic acid(s) that are detected preferentially have nucleotide sequences that are complementary to the nucleic acid sequences of the corresponding probe(s) to which they specifically bind (hybridize). The term target nucleic acid may refer to the specific subsequence of a larger nucleic acid to which the probe specifically hybridizes, or to the overall sequence (e.g., gene or mRNA) whose abundance (concentration) and/or expression level it is desired to detect. The difference in usage will be apparent from context.

The term “cross-linking” when used in reference to cross-linking nucleic acids refers to attaching nucleic acids such that they are not separated under typical conditions that are used to denature complementary nucleic acid sequences. Crosslinking preferably involves the formation of covalent linkages between the nucleic acids. Methods of cross-linking nucleic acids are described herein.

The phrase “coupled to a support” means bound directly or indirectly thereto including attachment by covalent binding, hydrogen bonding, ionic interaction, hydrophobic interaction, or otherwise.

“Transcribing a nucleic acid” means the formation of a ribonucleic acid from a deoxyribonucleic acid and the converse (the formation of a deoxyribonucleic acid from a ribonucleic acid). A nucleic acid can be transcribed by DNA-dependent RNA polymerase, reverse transcriptase, or otherwise.

A labeled moiety means a moiety capable of being detected by the various methods discussed herein or known in the art.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The phrase “hybridizing specifically to”, refers to the binding, duplexing, or hybridizing of a molecule preferentially to a particular nucleotide sequence under stringent conditions when that sequence is present in a complex mixture (e.g., total cellular) DNA or RNA. The term “stringent conditions” refers to conditions under which a probe will hybridize preferentially to its target subsequence, and to a lesser extent to, or not at all to, other sequences. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH. The Tₘ is the temperature (under defined ionic strength, pH, and nucleic acid concentration) at which 50% of the probes complementary to the target sequence hybridize to the target sequence at equilibrium. (As the target sequences are generally present in excess, at Tₘ, 50% of the probes are occupied at equilibrium). Typically, stringent conditions will be those in which the salt concentration is at least about 0.01 to 1.0 M Na ion concentration (or other salts) at pH 7.0 to 8.3 and the temperature is at least about 30.degree. C. for short probes (e.g., 10 to 50 nucleotides). Stringent conditions may also be achieved with the addition of destabilizing agents such as formamide.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.” In the case of expression monitoring arrays, perfect match probes are typically preselected (designed) to be complementary to particular sequences or subsequences of target nucleic acids (e.g., particular genes). In contrast, in generic difference screening arrays, the particular target sequences are typically unknown. In the latter case, perfect match probes cannot be preselected. The term perfect match probe in this context is used to distinguish that probe from a corresponding “mismatch control” that differs from the perfect match in one or more particular preselected nucleotides as described below.

The term “mismatch control” or “mismatch probe”, in expression monitoring arrays, refers to probes whose sequence is deliberately selected not to be perfectly complementary to a particular target sequence. For each mismatch (MM) control in a high-density array there preferably exists a corresponding perfect match (PM) probe that is perfectly complementary to the same particular target sequence. In “generic” (e.g., random, arbitrary, haphazard, etc.) arrays, since the target nucleic acid(s) are unknown perfect match and mismatch probes cannot be a priori determined, designed, or selected. In this instance, the probes are preferably provided as pairs where each pair of probes differ in one or more preselected nucleotides. Thus, while it is not known a priori which of the probes in the pair is the perfect match, it is known that when one probe specifically hybridizes to a particular target sequence, the other probe of the pair will act as a mismatch control for that target sequence. It will be appreciated that the perfect match and mismatch probes need not be provided as pairs, but may be provided as larger collections (e.g., 3, 4, 5, or more) of probes that differ from each other in particular preselected nucleotides. While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions. In a particularly preferred embodiment, perfect matches differ from mismatch controls in a single centrally-located nucleotide.

The terms “background” or “background signal intensity” refer to hybridization signals resulting from non-specific binding, or other interactions, between the labeled target nucleic acids and components of the oligonucleotide array (e.g., the oligonucleotide probes, control probes, the array substrate, etc.). Background signals may also be produced by intrinsic fluorescence of the array components themselves. A single background signal can be calculated for the entire array, or a different background signal may be calculated for each region of the array. In a preferred embodiment, background is calculated as the average hybridization signal intensity for the lowest 1% to 10% of the probes in the array, or region of the array. In expression monitoring arrays (i.e., where probes are preselected to hybridize to specific nucleic acids (genes)), a different background signal may be calculated for each target nucleic acid. Where a different background signal is calculated for each target gene, the background signal is calculated for the lowest 1% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g. probes directed to nucleic acids of the opposite sense or to genes not found in the sample such as bacterial genes where the sample is of mammalian origin). Background can also be calculated as the average signal intensity produced by regions of the array that lack any probes at all.

The term “quantifying” when used in the context of quantifying nucleic acid abundances or concentrations (e.g., transcription levels of a gene) can refer to absolute or to relative quantification. Absolute quantification may be accomplished by inclusion of known concentration(s) of one or more target nucleic acids (e.g. control nucleic acids such as BioB or with known amounts the target nucleic acids themselves) and referencing the hybridization intensity of unknowns with the known target nucleic acids (e.g. through generation of a standard curve). Alternatively, relative quantification can be accomplished by comparison of hybridization signals between two or more genes or between two or more treatments to quantify the changes in hybridization intensity and, by implication, transcription level.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g. nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Methods of alignment of sequences for comparison are well known in the art. Optimal alignment of sequences for comparison may be conducted by the local homology algorithm of Smith and Waterman, Adv. Appl. Math. 2: 482 (1981), by the homology alignment algorithm of Needleman and Wunsch J. Mol. Biol. 48: 443 (1970), by the search for similarity method of Pearson and Lipman, Proc. Natl. Acad. Sci. USA 85: 2444 (1988), by computerized implementations of these algorithms (including, but not limited to CLUSTAL in the PC/Gene program by Intelligenetics, Mountain View, Calif., GAP, BESTFIT, FASTA, and TFASTA in the Wisconsin Genetics Software Package, Genetics Computer Group (GCG), 575 Science Dr., Madison, Wis., USA), or by inspection. In particular, methods for aligning sequences using the CLUSTAL program are well described by Higgins and Sharp in Gene, 73: 237-244 (1988) and in CABIOS 5: 151-153 (1989)).

For gene expression analyses, the basic hybridization and detection principles used are similar for both gene chip or microsphere (bead) based technologies. For example, target specific complementary oligonucleotides, usually in the length of 25-60 bases, are attached at defined locations on a chip or to specific color-coded microspheres and then used to profile nonradioisotopically labeled nucleic acid samples. This is done through standard hybridization reactions and subsequent fluorescent detection of the labeled nucleic acid probe that has hybridized to its respective complementary sequence. While the gene chip technology is better suited to covering thousands of genes at a time, the microsphere (beads) based technologies offer greater flexibility in the choice of which genes are to be assayed in any given experiment.

Biotin, in part, due to its high affinity for streptavidin or avidin, has been the most widely used nonradioistopic label for nucleic acids for more than twenty years now. There are essentially two basic methods for labeling nucleic acids with biotin: enzymatic or chemical. Langer, Waldrop and Wald [Langer et al., “Enzymatic synthesis of biotin-labeled polynucleotides: novel nucleic acid affinity probes. Proc. Natl. Acad. Sci. USA, 78:6633-6637 (1981)] introduced the preparation of biotin-labeled probes by enzymatic incorporation of biotin-labeled analogs of dUTP and UTP into nucleic acids. Despite the success of this method and widespread availability of labeling kits, it has the disadvantage of requiring expensive enzymatic and analog reagents.

A cisplatin chemical linkage based nucleic acid labeling reagent [see U.S. Pat. No. 6,338,943 issued to Houthoff et al.] is also commercially available (Kreatech Biotechnology, Amsterdam, The Netherlands). That reagent labels DNA and RNA by binding of a platinum complex with a detectable molecule, i.e. biotin, to the N7 position of guanine. Again, however, the reagent costs make it impractical for large scale, high throughput usage.

Photobiotin, which is a photo-activatable analog of biotin [Forster et al., “Non-radioactive Hybridization Probes Prepared by the Chemical Labeling of DNA and RNA with a Novel Reagent, Photobiotin,” Nucleic Acids Res., 13:745-761 (1985) and U.S. Pat. No. 489,895 issued to Symons] has also been shown to provide a simple, rapid and direct reagent for labeling nucleic acids [see Habili et al., “Nonradioactive, Photobiotin-labelled DNA Probes for the Routine Diagnosis of Barley Yellow Dwarf Virus,” J. Virol. Methods, 16:225-237 (1987), Khan, A. M. and Wright, P. J., “Detection of flavivirus RNA in Infected Cells Using Photobiotin-Labelled Hybridization Probes,” J. Virol. Methods, 15:121-130 (1987), Szentirmay et al., “Demonstration by In Situ Hybridization of Ret Proto-Oncogene mRNA in Developing Placenta During Mid-Term of Rat Gestation,” Oncogene, 5:701-705 (1990), McInnes et al., “Preparation and Uses of Photobiotin,” Methods in Enzymology, 184:588-600 (1990), Hilario, E., “Photobiotin Labeling,” Methods Mol. Biol., 179:11-22 (2002) and Weise et al., “Fluorescence In Situ Hybridization (FISH) on Human Chromosomes using Photoprobe Biotin-Labeled Probes,” J. Histochem. Cytochem., 51:549-551 (2003)].

In contrast to the other labeling methods, photobiotin is inexpensive and is also commercially available as PHOTOPROBE® Biotin (Vector Lab. Inc., Burlingame, Calif.). Basically, photobiotin is biotin attached by a charged linker arm to a photoreactive arylazide group. Several forms are available, which differ either in the length or type of the linker arm. In all cases, when a mixture of photobiotin and nucleic acids is exposed to a strong visible light under defined conditions, the arylazide group is converted to an extremely reactive arylnitrene, which allows the formation of linkages to nucleic acid. The linkage is stable under standard hybridization conditions and is presumably covalent. As an alternative to using strong visible light for the conversion of the arylazide group to the reactive arynitrene required for nucleic acid labeling, it has been shown that exposure of an arylazide coupling group to heat instead works equally as well [Daniel et al., “FastTag™ Nucleic Acid Labeling System: A Versatile Method for Incorporating Haptens, Fluorochromes and Affinity Ligands into DNA, RNA and Oligonucleotides,” BioTechniques, 24:484-489 (1998)].

Anti-sense RNA (aRNA) is a RNA molecule transcribed off of the coding strand of DNA. This is complementary to the sense messenger RNA (mRNA) and is used for array hybridizations. Array hybridizations require large amounts of labeled aRNA that often needs to be made from very limited amounts of initial messenger RNA (mRNA). In order to accomplish this, RNA amplification methods have been developed and commercialized. For the most part, these are based on either linear amplification, PCR or a combination of both. The T7 polymerase based linear amplification procedure [see Van Gelder et al., “Amplified RNA Synthesized from Limited Quantities of Heterogeneous cDNA,” Proc. Natl. Acad. Sci. USA, 87:1663-1667(1990) and U.S. Pat. No. 5,716,785 and U.S. Pat. No. 5,891,636 issued to Van Gelder et al.] appears to be one of the most widely used and is the basis of synthesis kits available through, for example, Affymetrix (Santa Clara, Calif.), Agilent Technologies (Palo Alto, Calif.) and Ambion (Austin, Tex.). In brief, that procedure entails, a first strand cDNA synthesis by reverse transcription from mRNA using a T7 oligo(dT) primer with a T7 promoter sequence, followed by a second strand cDNA synthesis, the double-stranded cDNA then serves as the template for an in vitro transcription reaction where multiple copies of aRNA are generated.

In general, approximately 5-10 μg of labeled aRNA is required for each multiplexed array hybridization, regardless of type, i.e. chip based or microsphere (bead) based. Based on current list prices, the use of photobiotin as described in this invention, reduces the aRNA labeling costs per μg aRNA by up to approximately 94% in comparison to the use of biotin-labeled analogs or other chemical linkage based nucleic acid labeling reagents.

When used in conjunction with a multiplex capable system, such as the Luminex 100 fluidic type instrument and xMAP® color-coded microspheres, the present invention provides a method for cost effective, high throughput, multiplexed nucleic acid (gene) expression analysis.

The invention is further illustrated by the following non-limiting examples.

### EXAMPLES

### Example 1

### aRNA Amplification

MessageAmp™ aRNA kits (Ambion Inc., Austin, Tex.) were used for RNA amplification, i.e. synthesize of aRNA. A modified protocol was developed for use in conjunction with a subsequent 96-well format photobiotin labeling procedure. The modifications included reducing the reactions volumes to ⅖ of the original size and incorporating 96-well gel filtration plates for 2 purification steps. The aRNA amplification procedure is a five-step process comprised of 1.) a first strand cDNA synthesis, 2.) a second strand cDNA synthesis, 3.) a cDNA purification, 4.) an in vitro transcription which the generates multiple copies of aRNA and 5.) an aRNA purification.

A) First Strand cDNA Synthesis (8 μl Reaction)

Approximately 1-2 μg of total RNA was placed per well in a sterile RNase-free 96-well PCR plate or PCR microfuge tube to which 1 μl of T7 Oligo(dT) primer was added and brought to a final volume of 4.8 μl with nuclease-free water.

The mixture was incubated for 10 min at 70° C. in a thermal cycler. The RNA samples were then removed from the 70° C. incubator, centrifuged briefly (˜5 sec) to collect samples at the bottom of plate/tube and placed on ice while preparing the reverse transcription master mix. In a separate tube, enough reverse transcription master mix was prepared to synthesize first strand cDNA from all the RNA samples. This included a 5% overage to cover pipeting error. The following recipe is for a single reaction: 0.8 μl 10X First Strand Buffer, 0.4 μl ribonuclease inhibitor, 1.6 μl dNTP mix and 0.4 μl reverse transcriptase. This was mixed well by gentle pipeting up and down, centrifuged briefly (˜5 sec) to collect the master mix at the bottom of the tube and placed on ice. 3.2 μl reverse transcription master mix was added to each RNA sample, mixed well by gentle pipeting up and down and centrifuged briefly (˜5 sec) to collect the mix at the bottom of the tube/well. The reaction mixtures were incubated for 2 hr at 42° C. in a thermal cycler. After incubation, the reaction mixtures were centrifuged briefly (˜5 sec) to the collect samples at the bottom of the plate/tube. The plate/tube were placed on ice for second strand cDNA synthesis.

B) Second Strand cDNA Synthesis (40 μl Reaction)

In a separate tube on ice, the second strand cDNA synthesis reagent master mix was prepared for all samples. This included a 5% overage to cover pipeting error. The following recipe is for a single reaction: 25.2 μl nuclease-free water, 4 μl 10X Second Strand Buffer, 1.6 μl dNTP Mix 0.8 μl DNA Polymerase, and 0.4 RNase H. 32 μl second strand CDNA synthesis master mix was added to each 8 μl first strand cDNA sample, mixed by gently pipeting and centrifuged briefly (˜5 sec) to collect the samples at the bottom of the plate/tube. The reaction mixture was incubated for 2 hr at 16° C. This was immediately followed with the cDNA purification.

C) cDNA Purification

For the cDNA purification, EdgeBioSystems Performa™ DTR 96-well gel filtration plates and QuickStep2 SOPE (solid-phase oligo/protein elimination) resin were used (Edge BioSystems, Gaithersburg, Md.). The SOPE™ resin was mixed by briefly vortexing and 8 μl SOPE™ Resin was added directly to each 40 μl cDNA reaction, mixed well and let stand at room temperature while preparing the Performa DTR 96-well gel filtration plate. The adhesive plate sealers were removed from top and bottom of the Performa™ DTR 96-well gel filtration plate, the plate was covered with a lid and stacked on top of a 96-well flat-bottom microplate supplied with kit. The assembly was placed in a cushioned centrifuge plate carrier and centrifuged for 2 minutes at 750×g. The SOPE™/cDNA reaction mixture was then transferred by slowly pipeting directly to the wells of the Performa™ DTR 96-well gel filtration plate and covered with a lid. The Performa™ DTR 96-well gel filtration plate was stacked on top of the 96-well Performa™ DTR 96-well gel filtration plate-bottom microplate supplied with the kit, the assembly placed in a cushioned centrifuge plate carrier and centrifuged for 2 minutes at 750×g. The eluates were retained and reduced to a volume of 6.4 μL in a SpeedVac.

D) In vitro Transcription to Synthesize aRNA (16 μl Reaction)

In a separate tube at room temp, the transcription reaction components master mix was prepared for all samples. This included a 5% overage to cover pipeting error. The following recipe is for a single reaction: 1.6 μl T7 ATP Solution (75 mM), 1.6 μl T7 CTP Solution (75 mM), 1.6 μl T7 GTP Solution (75 mM), 1.6 μl T7 UTP Solution (75 mM), 1.6 μl T7 10X Reaction Buffer, and 1.6 μl T7 Enzyme Mix. 9.6 μl master mix was added to each well containing 6.4 μl purified cDNA. The plate was sealed and mixed gently, followed by a brief centrifugation (˜5 sec) to collect reactions at bottom of wells. The reactions were incubated for 14 hr at 37° C. in a water bath after which 0.8 μl Dnase I was added to each reaction to remove template cDNA from the aRNA. The reactions were then mixed gently, centrifuged briefly and incubated 30 minutes at 37° C. before proceeding to the aRNA purification step.

E) aRNA Purification

For the aRNA purification, EdgeBioSystems Performa™ SR 96-well gel filtration plates were used (Edge BioSystems, Gaithersburg, Md.). The aRNA reaction volumes were brought up to 50 μl with RNase free dH₂O. The top and bottom adhesive tapes were removed from a Performa™ SR 96-well plate, the plate covered with lid and stacked on top of a 96-well flat bottom plate. The assembly was placed on a cushioned centrifuge carrier designed to hold deep-well 96-well plates and centrifuged for 3 minutes at 850×g. The eluate was discarded. The SR 96-Well Plate was then stacked on top of a 96-well V-bottom plate and the samples carefully applied to the center of each well, without piercing the gel bed or touching the sides of the well and the plate was then covered with lid. The assembly was placed on a cushioned centrifuge carrier designed to hold deep-well 96-well plates and centrifuged for 3 minutes at 850×g. The eluates were retained and the concentrations determined using a spectrophotometer.

### Example 2

### Photobiotin Labeling of aRNA

Photobiotin is commercially available as PHOTOPROBE® Biotin (Vector Lab., Burlingame, Calif.). It was supplied as a dry powder and was stored at −20° C. to −80° C. until reconstituted. 0.5 mg PHOTOPROBE® (long arm) Biotin was reconstituted with 500 μl of distilled water, followed by gentle mixing (1 μg/μl end concentration). The solution was stored at −20° C. to −80° C, protected from light. Under these conditions, the solution is reported to be stable for up to 1 year. For aRNA labeling with PHOTOPROBE® Biotin, PHOTOPROBE® Biotin:aRNA (w:w) ratios of between 1:5 and 1:25 were primarily used. For each reaction, 10 μg of aRNA was brought up to 10 μl in TE per well in a sterile RNase-free 96-well PCR plate. An appropriate amount of PHOTOPROBE® Biotin was brought up to 10 μl in TE and added to each well and mixed by gentle pipeting. The mixture was incubated for 30-60 min. at 95° C. in a thermal cycler. After the incubation, the plate was centrifuged briefly (˜5 sec) to collect samples at bottom and placed on ice. The PHOTOPROBE® Biotin labeled aRNA was purified using an Edge BioSystems Performa™ DTR 96-well gel filtration plate. For this, the adhesive plate sealers were removed from the top and bottom of a Performa™ DTR 96-well gel filtration plate and covered with a lid. The Performa™ DTR 96-well gel filtration plate was stacked on top of a 96-well flat-bottom microplate supplied with the kit and the assembly placed in a cushioned centrifuge plate carrier and centrifuged for 2 minutes at 750×g. The aRNA PHOTOPROBE® Biotin reaction mixture was transferred by pipeting to the Performa DTR 96-well gel filtration plate. The Performa™ DTR 96-well gel filtration plate was stacked on top of the 96-well Performa™ DTR 96-well gel filtration plate-bottom microplate supplied with the kit, the assembly was then placed in a cushioned centrifuge plate carrier and centrifuged for 2 minutes at 750×g. The eluates were retained for hybridization.

### Example 3

### Coupling of Gene Specific Oligonucleotides to Microspheres

XMAP™ multi analysis carboxylated microspheres were obtained from Luminex Corporation (Austin, Tex.). The stock microspheres were resuspended by vortexing and sonication. 2.5×10⁶ (200 μL) of each stock of microspheres used were transferred to microfuge tubes and pelleted by microcentrifugation at ≧8000×g for 1-2 minutes. The supernatant was removed and the pelleted microspheres resuspended in 50 μL of 0.1M MES, pH 4.5, by vortexing and sonication. 0.2 nanomole of 5′ amine Uni-Link gene specific capture oligos (1 μL of 1 mM stock) were added to the resuspended microspheres and mixed by vortexing. A fresh solution of 10 mg/mL EDC (1-Ethyl-3-(3-dimethylaminopropyl)carbodimide HCl, Pierce, Rockford, Ill.) was prepared in dH₂O and 2.5 μL of it was added to the microspheres followed by vortexing and a 30 minutes incubation at room temperature in the dark. A second, fresh solution of 10 mg/mL EDC was then prepared in dH₂O and 2.5 μL of it was added to the microspheres, followed by vortexing and another 30 minute incubation at room temperature in the dark. This was repeated a third time after which two wash steps followed. For the first, 1 mL of 0.02% Tween® 20 was added to the coupled microspheres with mixing by vortexing. The coupled microspheres were pelleted, the supernatant removed and the microspheres resuspended in 1 mL of 0.1% SDS by vortexing. The coupled microspheres were pelleted again by centrifugation and resuspended in 100 μL of TE, pH 8.0 by vortexing and sonication for approximately 20 seconds. The coupled microspheres were enumerated using a hemacytometer and stored at 4° C. in the dark until used.

### Example 4

### Hybridization of Photobiotin Labeled aRNA to Gene Specific Oligo-Coupled Beads

5-10 μg of photobiotin labeled aRNA in 20 μl TE was heated at 94° C. for 5-10 minu for each reaction. The samples were then centrifuged briefly (˜5 sec) to collect the samples at the bottom of a 96-well plate. Note: that step was only done if the aRNA had been frozen and stored after labeling. 40 μl of gene specific oligo-conjugated beads (2,500-5,000 beads per gene) pre-mixed in 1.5×TMAC hybridization buffer (4.5 M TMAC/75 mM Tris-HCL, pH 8.0/6 mM EDTA, pH 8.0/0.15% Sarkosyl) was added to each well of denatured photobiotin labeled aRNA and mixed well on a plate shaker (i.e. use a Labnet Orbit P2 96-well plate shaker for plates, 20-45 seconds at ˜1,100 rpm). This was followed by a 4 hrs—O/N incubation at 60° C. in a hybridization incubator shaker with a shaking speed of ˜200-300 rpm and protected from light.

After the incubation, the beads/hybridization mixtures were transferred to a Millipore MultiScreen®-BV 96-well plate for washes and Luminex analysis. The hybridization buffer was carefully removed using a Millipore 96-well plate vacuum set-up. The beads were washed 3 times using 50-75 μl of 1×PBS/1 mM EDTA/0.01% Tween® 20 (wash/detection buffer) per well. The Millipore 96-well plate vacuum set-up was used to remove the wash buffer each time. The beads were resuspended after each wash using a Labnet Orbit P2 96-well plate shaker for ˜20-45 seconds at ˜1,100 rpm. After the last wash, the beads were resuspended in 75 μl of streptavidin-phycoerythrin conjugate (Prozyme PJ31S) diluted 1:500 in 1×PBS/1 mM EDTA/0.01% Tween® 20 (wash/detection buffer) and incubated, protected from light (plate covered with aluminum foil), for 15 minutes on a Labnet Orbit P2 96-well plate shaker set at 300 rpm. The Millipore 96-well plate vacuum set-up was used to remove the excess streptavidin-phycoerythrin conjugate and the solution was then replaced with 75 μl of wash/detection buffer. The beads were resuspended using a Labnet Orbit P2 96-well plate shaker for ˜20-45 seconds at ˜1,100 rpm and then analyzed on a Luminex 100 fluidic type instrument.

### Example 5

### Reproducibility of Gene Expression Detection Using Photobiotin Labeled aRNA from Independent Reactions

To examine the reproducibility of multiplex hybridization and detection using photobiotin labeled aRNA, labeling, hybridization, and detection reactions were set up in triplicate following the procedures detailed in materials and methods. For this, five human genes were chosen for the multiplex reactions with three gene specific oligonucleotides being used for each gene. Each gene specific set of oligonucleotides was conjugated to specific color-coded xMAP® multi-analyte COOH microspheres as described in materials and methods. The five genes were:

| | |
|---|---|
| 1) | Glyceraldehyde-3-phosphate dehydrogenase (Gene |
| | Name: GAPDH), GenBank mRNA Accession Number |
| | NM_002046, using GAPDH specific |
| | oligonucleotides: zc45814 (gttgccatgtagaccccttg |
| | aagag) (SEQ ID NO:1), zc45815 (accagccccagcaaga |
| | gcacaagag) (SEQ ID NO:2), zc45816 (tgacttcaacag |
| | cgacacccactcc) (SEQ ID NO:3); |
| 2) | Peptidyl-prolyl isomerase A (Cyclophilin A) |
| | (Gene Name: PPIA) GenBank Accession Number |
| | NM_021130, using PPIA specific oligo- |
| | nucleotides: zc45811 (acagaattattccagggtttatg |
| | tg) (SEQ ID NO:4), zc45812 (gcagtatcctagaatcttt |
| | gtgctc) (SEQ ID NO:5), zc45813 (tgagaacttcatcct |
| | aaagcatacg) (SEQ ID NO:6); |
| 3) | Ribosomal protein L13a (Gene Name: RPL13A), |
| | GenBank mRNA Accession Number NM_012423, using |
| | RPL13A specific oligonucleotides: zc45628 |
| | (gcccatgctcctcacctgtattttg) (SEQ ID NO:7), |
| | zc45629 (actcggagaattgtgcaggtgtcat) |
| | (SEQ ID NO:8), zc45630 (ccagttactatgagtgaaaggga |
| | gc) (SEQ ID NO:9); |
| 4) | Glucuronidase, beta (Gene Name: GUSB), GenBank |
| | mRNA Accession Number NM_000181, using GUSB |
| | specific oligonucleotides: zc45631 (gcctgggtttt |
| | gtggtcatctattc) (SEQ ID NO:10), zc45632 (gacga |
| | gagtgctggggaataaaaag) (SEQ ID NO:11), zc45633 |
| | (tatcagaagcccattattcagagcg) (SEQ ID NO:12); |
| and | |
| 5) | Hypoxanthine phosphoribosyltransferase 1 (Gene |
| | Name: HPRT1), GenBank mRNA Accession Number |
| | NM_012423, using HPRT1 specific oligonucleo- |
| | tides: zc47406 (agcaaaatacaaagcctaagatgag) |
| | (SEQ ID NO:13), zc47407 (tgggcggattgttgtttaact |
| | tgta) (SEQ ID NO:14), zc47408 (gttagaaaagtaaga |
| | agcagtcaat) (SEQ ID NO:15). |

These genes are among those often used as reference or control genes because of their relative constitutive expression and are commonly referred to as housekeeping genes. The genes selected are expressed at various levels from high (i.e., GAPDH) to low (i.e., HPRT).

The results of the multiplex hybridization and gene expression detection reactions using photobiotin labeled aRNA from human universal reference total RNA (BD Biosciences Clontech, Palo Alto, Calif.) and the five genes described above (GAPDH, PPIA, RPL13A, GUSB, HPRT) yielded the following coefficients of variance (CV %) for the individual genes (average of independent triplicates): GAPDH (5.3%), PPIA (7.3%), RPL13A (10.9%), GUSB (8.0%), HPRT (9.0%). The results indicate that using photobiotin for labeling of anti-sense RNA (aRNA) as hybridization probe in conjunction with a multiplex capable system such as the Luminex® 100 fluidic type instrument and xMAP® color-coded microspheres is highly reproducible for multiplex gene expression detection.

### Example 6

### Comparison of Gene Expression Detection Carried Out with aRNA Made Using Biotin Labeled NTPs and aRNA Labeled with Photobiotin

To examine the effects of photobiotin aRNA labeling on gene expression detection, three parallel multiplex hybridization and detection reactions were set up in duplicate using aRNA made from human universal reference total RNA (Clontech, Palo Alto, Calif.) that was labeled with either photobiotin, biotin-16-UTP or both biotin-11-CTP and biotin-16-UTP. The labeling of aRNA with photobiotin was carried out following the procedures in materials and methods. The labeling of aRNA with biotin-16-UTP or both biotin-11-CTP and biotin-16-UTP was carried out using a MessageAmp™ aRNA kit (Ambion Inc., Austin, Tex.) according to the manufactures instructions.

Ten human genes were chosen for the multiplex reactions with one specific oligonucleotide being used for each gene. Each gene specific oligonucleotide had an additional 10-base long non-human sequence added to the 5′ end for quality control. The genes selected are among those often used as reference or control genes because of their relative constitutive expression and are commonly referred to as housekeeping genes. The gene specific oligonucleotides were conjugated to specific color-coded xMAP® multi-analyte COOH microspheres as described in materials and methods. The ten genes were:

| | |
|---|---|
| 1) | Beta-actin (Gene Name: ACTB), GenBank mRNA |
| | Accession Number NM_001101, using ACTB specific |
| | oligonucleotide: zc52566 (cagccgaagtggaggtgatag |
| | cattgctttcgtgt) (SEQ ID NO:16); |
| 2) | Clathrin heavy polypeptide (Hc) (Gene Name: |
| | CLTC), GenBank mRNA Accession Number NM_004859, |
| | using CLTC specific oligonucleotide: zc52567 |
| | (cagccgaagtcccttatgttgtgctgtatcctgtg) |
| | (SEQ ID NO:17); |
| 3) | Glyceraldehyde-3-phosphate dehydrogenase (Gene |
| | Name: GAPDH), GenBank mRNA Accession Number |
| | NM_002046, using GAPDH specific oligonucleo- |
| | tide: zc52568 (cagccgaagtgttgccatgtagaccccttgaa |
| | gag) (SEQ ID NO:18); |
| 4) | Beta-glucuronidase (Gene Name: GUSB), GenBank |
| | mRNA Accession Number NM_000181, using GUSB |
| | specific oligonucleotide: zc52569 (cagccgaagtgc |
| | ctgggttttgtggtcatctattc) (SEQ ID NO:19); |
| 5) | Hypoxanthine guanine phosphoribosyltransferase |
| | 1 (Gene Name: HPRT), GenBank mRNA Accession |
| | Number NM_000194, using HPRT specific oligo- |
| | nucleotide: zc52570 (cagccgaagttgggcggattgttgtt |
| | taacttgta) (SEQ ID NO:20); |
| 6) | Cyclophilin A (Gene Name: PPIA), GenBank mRNA |
| | Accession Number NM_021130, using PPIA specific |
| | oligonucleotide: zc52571 (cagccgaagtagcctccgcct |
| | cctgggttcaagtg) (SEQ ID NO:21); |
| 7) | Ribosomal protein L13A (Gene Name: RPL13), |
| | GenBank mRNA Accession Number NM_001101, using |
| | RPL13 specific oligonucleotide: zc52572 (cagccg |
| | aagtgcccatgctcctcacctgtattttg) (SEQ ID NO:22); |
| 8) | Large ribosomal phosphoprotein P0 (Gene Name: |
| | RPLP0), GenBank mRNA Accession Number |
| | NM_001002, using RPLP0 specific oligo- |
| | nucleotide: zc52573 (cagccgaagtagccaaggaagagtcg |
| | gaggagtcg) (SEQ ID NO:23); |
| 9) | Transferrin receptor (Gene Name: TFRC), GenBank |
| | mRNA Accession Number NM_003234, using TFRC |
| | specific oligonucleotide: zc52574 (cagccgaagttg |
| | agattcctggttcgggtgttacg) (SEQ ID NO:24); |
| and | |
| 10) | Ubiquitin (Gene Name: UBC), GenBank mRNA |
| | Accession Number NM_021009, using UBC specific |
| | oligonucleotide: zc52575 (cagccgaagtcgagaatgtca |
| | aggcaaagatccaa) (SEQ ID NO:25). |

The gene expression ratios obtained from the multiplex hybridization and gene expression detection reactions using aRNA that was labeled with either photobiotin, biotin-16-UTP or both biotin-11-CTP and biotin-16-UTP and the ten genes described above (ACTB, CLTC, GAPDH, GUSB, HPRT, PPIA, RPL13, RPLP0, TFRC, and UBC) were (relative to RPLP0 and listed in the order of photobiotin labeled aRNA, both biotin-11-CTP and biotin-16-UTP labeled aRNA, just biotin-16-UTP labeled aRNA): ACTB (1.4, 1.2, 1.4), RPLP0 (1.0, 1.0, 1.0), GAPDH (4.2, 3.2, 2.7), GUSB (1.0, 1.1, 0.9), HPRT (0.2, 0.1, 0.1), PPIA (0.6, 0.8, 0.6), RPL13 (2.4, 2.5, 2.5), CLTC (1.1, 0.9, 0.9), TFRC (0.4, 0.3, 0.3), and UBC (2.2, 1.8, 2.8). The results show that the pattern of gene expression ratios was similar for the aRNA labeled with the photobiotin and the aRNAs with biotin labeled NTPs. This indicates that photobiotin can be used as an economical substitute for biotin labeled NTPs to make biotin labeled aRNA.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

What is claimed is:

1. A method of gene expression analysis, the method comprising using a high throughput system in conjunction with photobiotin-labeled aRNA hybridization probes.

2. The method of claim 1 wherein the high throughput system is s multiplex capable system.

3. A method of identifying differences between gene expression levels of two or more nucleic acid samples, each nucleic acid sample comprising a population of mRNA or nucleic acids; said method comprising the steps of:
(a) providing one or more photobiotin-labeled aRNA samples and capture probe arrays;
(b) hybridizing said nucleic acid samples to said one or more arrays to form hybrid duplexes between nucleic acids in said nucleic acid samples and probes in said one or more arrays; and
(c) determining a first value for a difference in hybridization between at least two probes differing from each other in at least one position to a first sample, and a second value for a difference in hybridization between the two probes to a second sample, a difference in hybridization values between said nucleic acid samples indicating a difference in a level of a nucleic acid between the first and second samples.

4. The method according to claim 3, wherein step (c) can involve between 2 and 100 probes.
